(19) United States
(12) Patent Application Publication
(10) Pub. No.: US 2026/0256578 A1
(43) Pub. Date: Sep. 3, 2026

(54) SKIRT ASSEMBLY FOR PROSTHETIC HEART VALVE

(71) Applicant: Edwards Lifesciences Corporation, Irvine, CA (US)

(72) Inventors: Aysan Rangchian, Rancho Palos Verdes, CA (US); Voleak Sambo, Long Beach, CA (US)

(21) Appl. No.: 19/664,388

(22) Filed: Apr. 30, 2026

Related U.S. Application Data
(63) Continuation of application No. PCT/US2024/055045, filed on Nov. 8, 2024.
(60) Provisional application No. 63/612,978, filed on Dec. 20, 2023.
(60) Provisional application No. 63/598,054, filed on Nov. 10, 2023.

(51) Int. Cl. A61F 2/24

(52) U.S. Cl. CPC A61F 2/2418; A61F 2/2415; A61F 2210/0076; A61F 2220/0075

(57) ABSTRACT

An implantable prosthetic device can include a radially expandable and compressible annular frame comprising a first end portion and a second end portion, an outer covering disposed on an outer surface of the frame, and an edge covering disposed on the first end portion of the frame. The edge covering can include a first layer disposed on an inner facing surface of the frame, and a second layer disposed on the outer surface of the frame. The edge covering is coupled to the frame via a first set of stitches extending through the first layer of the edge covering and the outer covering, and a second set of stitches extending through the first layer of the edge covering, the outer covering, and the second layer of the edge covering.

### CROSS REFERENCE TO RELATED APPLICATIONS

This application is a continuation of PCT Patent Application No. PCT/US2024/055045 filed on November 8, 2024, which claims the benefit of U.S. Provisional Application No. 63/598,054, filed November 10, 2023, and U.S. Provisional Application No. 63/612,978, filed December 20, 2023. The prior applications are incorporated herein by reference in their entirety.

### FIELD

The present disclosure relates to prosthetic heart valves, and to methods and assemblies for forming skirt assemblies and attaching the skirt assemblies to the frame of such prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Many expandable transcatheter heart valves comprise a metallic frames or stents. Such frames and stents can erode and/or damage the native anatomy over time. Accordingly, a need exists for prosthetic heart valves that do not damage the native anatomy over time.

### SUMMARY

Described herein are prosthetic heart valves, delivery apparatus, and methods for implanting prosthetic heart valves. The disclosed prosthetic heart valves, delivery apparatus, and methods can, for example, mitigate damage to the native valve during the implantation procedure and afterward. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, a prosthetic valve can comprise an outer covering disposed on an outer surface of the frame.

In some examples, a prosthetic valve can comprise an edge covering disposed on a first end portion of the frame.

In some examples, the edge covering can be coupled to the frame via a first set of stitches extending through a first layer of the edge covering and the outer covering and a second set of stitches extending through the first layer of the edge covering, the outer covering, and the second layer of the edge covering.

In some examples, the first set of stitches and a portion of the second set of stitches form a first suture line on a radially inwardly facing surface of the edge covering. In some examples, the first suture line is a solid suture line.

In some examples, a portion of the second set of stitches forms a second suture line on a radially outwardly facing surface of the edge covering. In some examples, the second suture line is a dashed suture line.

In some examples, a method for securing an edge covering to the frame of a prosthetic valve comprises coupling an edge covering to a frame using a first set of stitches extending through an outer covering and a first layer of the edge covering, the outer covering being disposed on an outer surface of the frame, and a second set of stitches extending through a second layer of the edge covering, the outer covering, and the first layer of the edge covering.

In some examples, the method further comprises, after sewing the first set of stitches, folding the edge covering in half lengthwise to form the first and second layers of the edge covering.

In some examples, a method comprises sterilizing any of the above described devices.

In some examples, a prosthetic heart valve comprises one or more of the components recited in Examples 1-23 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of a prosthetic heart valve, according to one example.

FIG. 2 is a side-elevation view of the prosthetic heart valve of FIG. 1.

FIG. 3 is a top plan view of the prosthetic heart valve of FIG. 1.

FIG. 4 is a perspective view of a step of a method for mounting an edge covering to a prosthetic heart valve, according to one example.

FIGS. 5A-5B are perspective views of another step of the method of FIG. 4, according to one example.

FIG. 6 is a perspective view of another step of the method of FIG. 4, according to one example.

FIGS. 7A-7B are perspective views of another step of the method of FIG. 4, according to one example.

FIGS. 8A-8B are perspective views of another step of the method of FIG. 4, according to one example.

FIGS. 9A-9B are perspective views of another step of the method of FIG. 4, according to one example.

FIGS. 10A-10B are perspective views of another step of the method of FIG. 4, according to one example.

FIGS. 11A-11B are perspective views of another step of the method of FIG. 4, according to one example.

FIGS. 12A-12B are perspective views of another step of the method of FIG. 4, according to one example.

FIGS. 13A-13B are perspective views of another step of the method of FIG. 4, according to one example.

FIG. 14 is a perspective view of another step of the method of FIG. 4, according to one example.

FIG. 15 is a perspective view of another step of the method of FIG. 4, according to one example.

FIGS. 16A-16C are perspective views of another step of the method of FIG. 4, according to one example.

FIGS. 17A-17B are perspective views of another step of the method of FIG. 4, according to one example.

FIGS. 18A-18B are perspective views of another step of the method of FIG. 4, according to one example.

FIGS. 19A-19B are perspective views of another step of the method of FIG. 4, according to one example.

FIGS. 20A-20B are perspective views of another step of the method of FIG. 4, according to one example.

FIG. 21 is a perspective views of another step of the method of FIG. 4, according to one example.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient’s body), while distal motion of the device is motion of the device away from the user and toward the implantation site (e.g., into the patient’s body). The terms “longitudinal” and “axial” refer to an axis extending in the proximal and distal directions, unless otherwise expressly defined.

### Examples of the Disclosed Technology

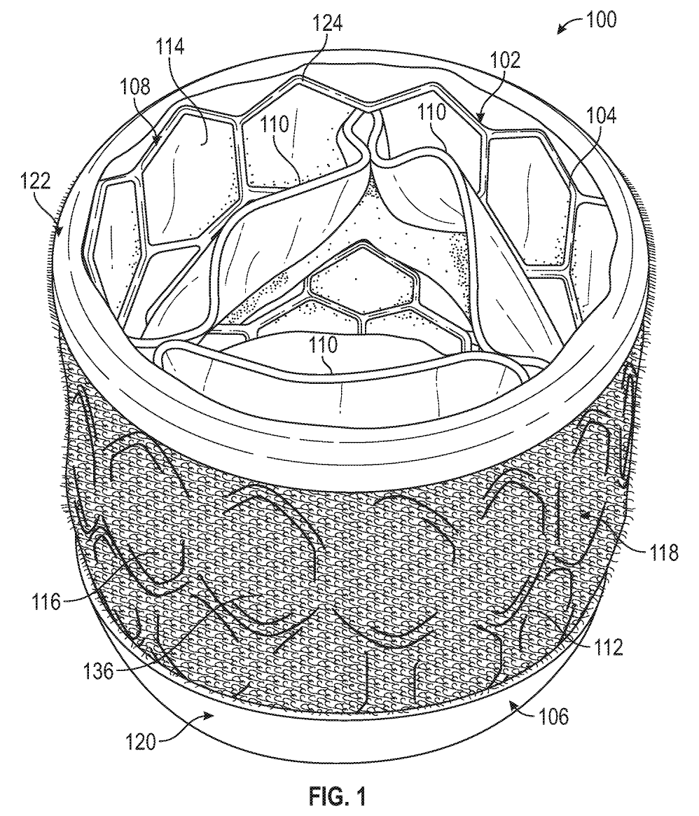

FIG. 1 shows an exemplary prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

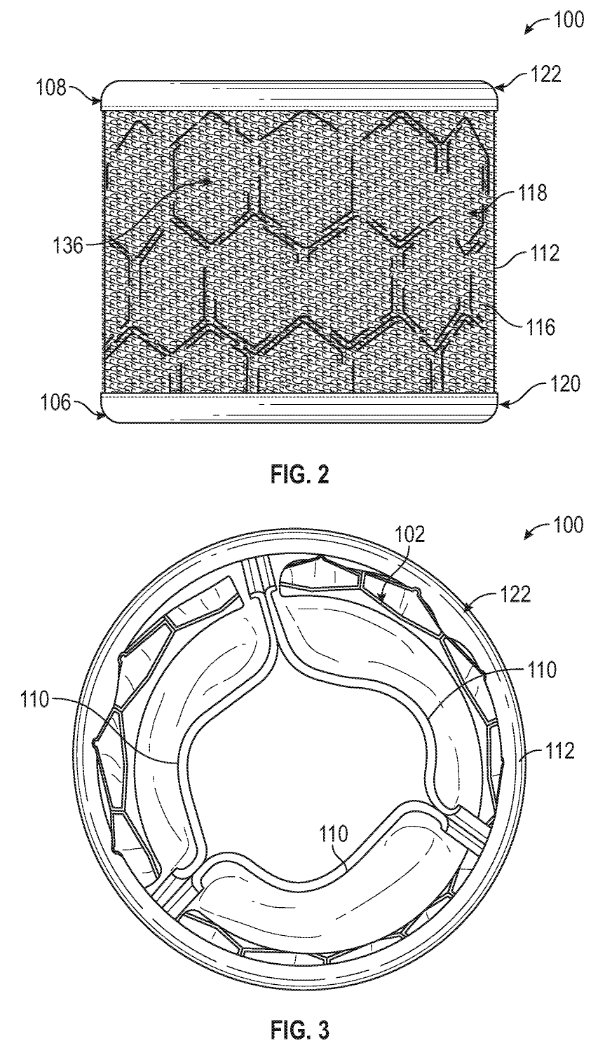

FIGS. 1-3 illustrate a representative example of a prosthetic heart valve 100. The prosthetic valve 100 includes a frame 102 formed by a plurality of angled strut members 104, and having an inflow end 106 and an outflow end 108. The prosthetic valve 100 also includes a leaflet structure comprising three leaflets 110 situated at least partially within the frame 102 and configured to collapse in a tricuspid arrangement similar to a native aortic valve, although the prosthetic valve alternatively can include two leaflets configured to collapse in a bicuspid arrangement in the manner of a native mitral valve, or more than three leaflets, as desired. The strut members 104 can form a plurality of apices 124 arranged around the inflow and outflow ends of the frame.

Suitable plastically expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. In some examples, when MP35N is used as the frame material, less material is needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

The prosthetic heart valve can include a covering or outer covering 112 configured to cushion (protect) native tissue in contact with the prosthetic valve after implantation, and to reduce damage to the tissue due to movement or friction between the tissue and surfaces of the valve. The covering 112 can also reduce paravalvular leakage. In the embodiment of FIG. 1, the covering 112 includes a first layer 114 configured as a backing layer, and a second layer 116 configured as a cushioning layer. The cushioning layer can be disposed on the backing layer, and can comprise a soft, plush surface 118 oriented radially outward so as to protect tissue or objects in contact with the cushioning layer. In the illustrated configuration, the covering 112 also includes an atraumatic inflow protective portion 120 extending circumferentially around the inflow end 106 of the frames, and an atraumatic outflow protective portion 122 extending circumferentially around the outflow end 108 of the frames. The portion of the cushioning layer 116 between the inflow and outflow protective portions 120, 122 can define a main cushioning portion 136. The first layer 114 and the second layer 116 can together form a sealing member or cover member that can be placed around the frame to form the covering 112. The sealing member/cover member can also comprise the protective portions 120, 122.

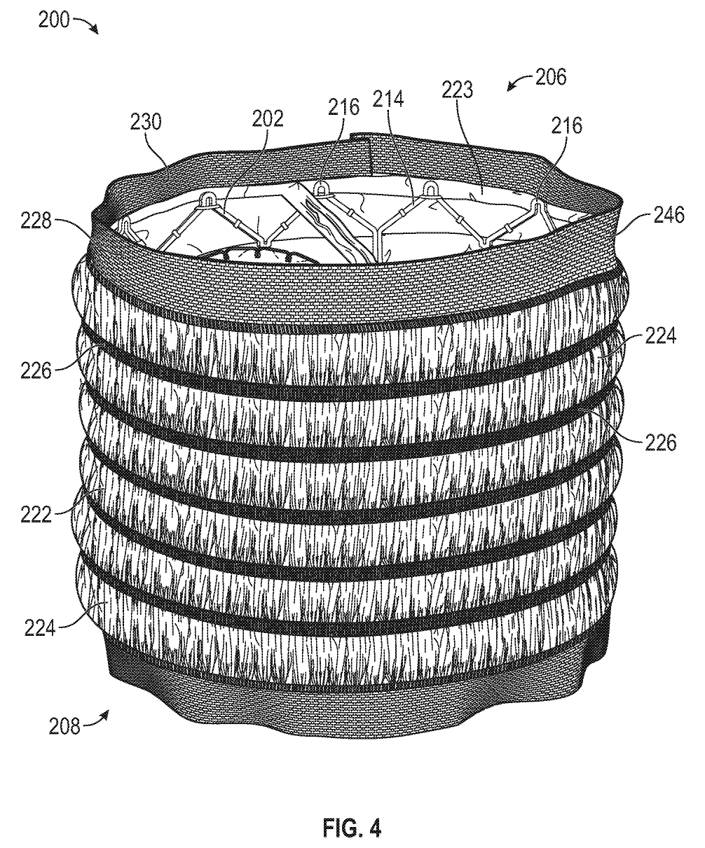

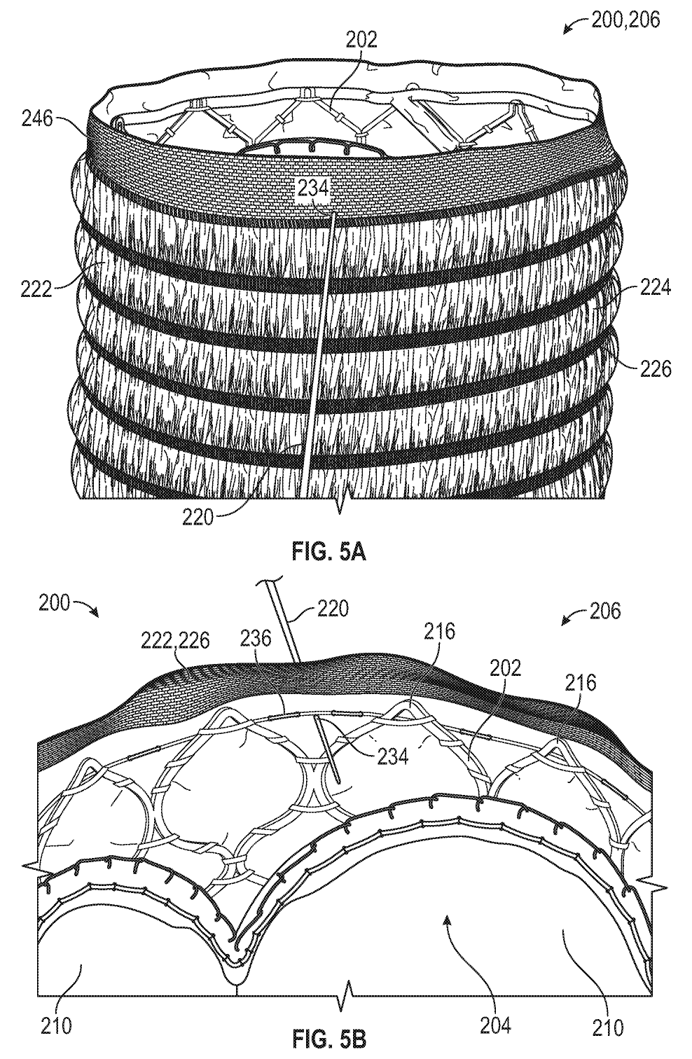

FIGS. 4-21 illustrate a prosthetic valve 200 similar to prosthetic valve 100 described previously. Prosthetic valve 200 is the same as prosthetic valve 100 except were indicated otherwise. That is, prosthetic valve 200 includes a frame 202, valvular structure 204 (FIG. 5B) comprising a plurality of leaflets 210, an inflow end portion 206, and an outflow end portion 208.

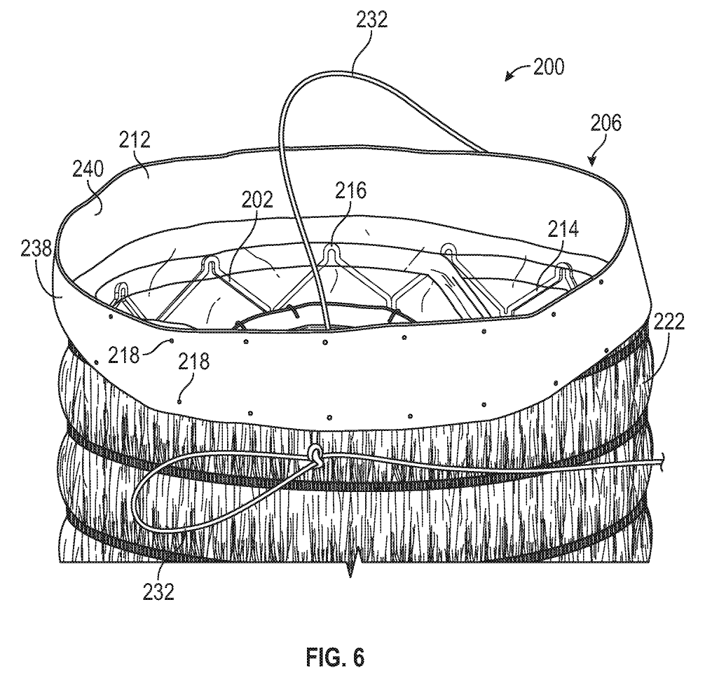

In some examples, in lieu of or in addition to the protective portions 120, 122 of prosthetic valve 100, the prosthetic valve 200 can comprise a skirt assembly comprising an outer covering 222 and one or more edge coverings 212 (FIG. 6). The one or more edge coverings 212 can be disposed on the inflow and/or outflow ends 206, 208 of the prosthetic valves 200. Though the edge coverings 212 and the method of attaching the edge coverings 212 to the prosthetic valve 200 are described with reference to the prosthetic valve 200, it should be understood that the edge coverings 212 and method of attachment can be used with any type of prosthetic valve, for examples, any of the prosthetic valves described in U.S. Publication Nos. 2017/0231756 and 2019/0000615, and PCT Publication No. WO2020/247907.

Each edge covering 212 can be configured to wrap around the inflow and/or outflow end 206, 208 of the prosthetic valve 200 (i.e., from an outer surface of the frame 202 to the inner surface 214). The edge covering 212 and outer covering 222 can thus cover the apices 216 of the frame 202 and prevent or mitigate contact between the apices 216 and the native anatomy when the prosthetic valve 200 is implanted within a patient.

The edge covering 212 can be wholly or partly formed of any suitable biological material, synthetic material (for example, any of various polymers), or combinations thereof. In some examples, the edge covering can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the edge covering 212 can comprise a fabric without interlaced yarns or fibers or randomly interlaced yarns or ribers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the edge covering 212 can comprise a non-textile or non-fabric material, such as in the form of a film or sheet, made from any of a variety of polymeric materials, such as PTFE, ePTFE, PET, polypropylene, polyamide, polyetheretherketone (PEEK), polyurethane (such as thermoplastic polyurethane (TPU)), etc. In some examples, the edge covering 212 can comprise a sponge material or foam, such as polyurethane foam. In some examples, the edge covering 212 can comprise natural tissue, such as pericardium (for example, bovine pericardium, porcine pericardium, equine pericardium, or pericardium from other sources).

In some examples, such as the illustrated example, the edge covering 212 can comprise preformed openings/apertures/holes 218 (FIG. 6) through which a needle can be sewn. In some examples, the edge covering 212 does not comprise preformed openings.

FIGS. 4-21 illustrate an exemplary method for attaching an outer covering 222 and an edge covering 212 to the inflow end 206 of a prosthetic valve 200. Though illustrated on the inflow end portion 206, it should be understood that this method can also be used to attach an edge covering 212 and the outer covering 222 to the outflow end 208 of the prosthetic valve 200. The described methods also advantageously fold the edge skirt 212 over the respective inflow or outflow end 206, 208 of the frames 202 during the sewing process, which simplifies and eases the attachment of the edge skirt 212 to the prosthetic valve and does not require that the user manually pinch the edge skirt 212 during the process.

The method generally comprises coupling an edge covering to an end portion of the frame of a prosthetic valve using a first set of stitches that extends through at least two layers of material (e.g., a first layer of the edge covering and an outer covering), and a second set of stitches that extends through at least three layers of material (e.g., the first layer of the edge covering, the outer covering, and a second layer of the edge covering). Such a method advantageously allows the first set of stitches to be sewn more easily (as passing through fewer layers of material requires less force on the needle) and advantageously allows the edge covering to be folded into two layers lengthwise during the attachment procedure by tightening the first set of stitches. This prevents the assembler from having to fold the edge covering prior to attachment, which can be difficult to accomplish.

Referring to FIG. 4, an outer covering 222 (which can also be referred to as an outer skirt) can be disposed on an outer surface of the frame 202 of the prosthetic valve 200. In some examples, the outer covering 222 can be disposed over an outer liner 223 (FIG. 4) coupled to the outer surface of the frame 202. The outer liner 223 can be coupled to the inflow end of the frame via a stitch line 236. In some examples, the prosthetic valve can further comprise an inner liner disposed on an inner surface of the frame 202. In some such examples, the inner liner can likewise be coupled to the frame via the stitch line 236.

The outer covering 222 can be the same as or similar to covering 112 described previously. In some examples, such as the illustrated example, the outer covering 222 can comprise a plurality of textured/plush portions 224 (also referred to as “floats” or “float portions”) disposed between a plurality of woven portions 226 including a woven portion 246 at the inflow end. The outer covering 222 can comprise an exterior surface 228 that faces away from a longitudinal axis of the prosthetic valve and an interior surface 230 that faces the longitudinal axis. Further details of the construction of the outer covering 222 are disclosed in U.S. Patent No. 11,654,023, which is incorporated herein by reference.

In some examples, the outer covering 222 is sized such that when it is placed around the frame 202, the woven portion 246 at the inflow end 206 of the prosthetic valves extends beyond the inflow end of the frame to form an extended portion of the covering 222 at the inflow end. Similarly, the outer covering 222 can include a woven portion 246 at the outflow end 208 of the prosthetic valves that extends beyond the outflow end of the frame to form an extended portion of the covering 222 at the outflow end.

Referring to FIGS. 5A-5B, a needle 220 with an attached suture 232 (FIG. 6) is inserted through the woven portion 246 of the outer covering 222 adjacent the inflow end 206 of the frame. FIG. 5A illustrates a view of outer diameter of the prosthetic valve 200 and FIG. 5B illustrates an inner diameter view during the same action. Throughout the application, figures designated with an “A” show the outer diameter view and figures designated with a “B” show the inner diameter view of the same action. As shown in FIG. 5B, the needle 220 can be inserted through the outer cover 222 from the exterior of the prosthetic valve to an interior at a first location 234 (also referred to as the “origin” or “origin point”) upstream of a circumferentially extending suture stitch line 236 (which can be, for example, a plurality of in-and-out stitches ) (FIG. 5B) that couples the outer liner 223 (FIG. 4) disposed between the outer covering 222 and the frame 202 to the frame 202.

As shown in FIG. 6, the edge covering 212 can be placed over the exterior surface of the woven portion 246 of the outer covering 222 at the inflow end 206 of the prosthetic valve 200. The edge covering 212 can comprise a first surface 238 and a second surface 240. In the orientation shown in FIG. 6 the first surface 238 is an exterior surface that faces away from a longitudinal axis of the prosthetic valve 200 and the second surface 240 is an interior surface that faces toward a longitudinal axis of the prosthetic valve 200.

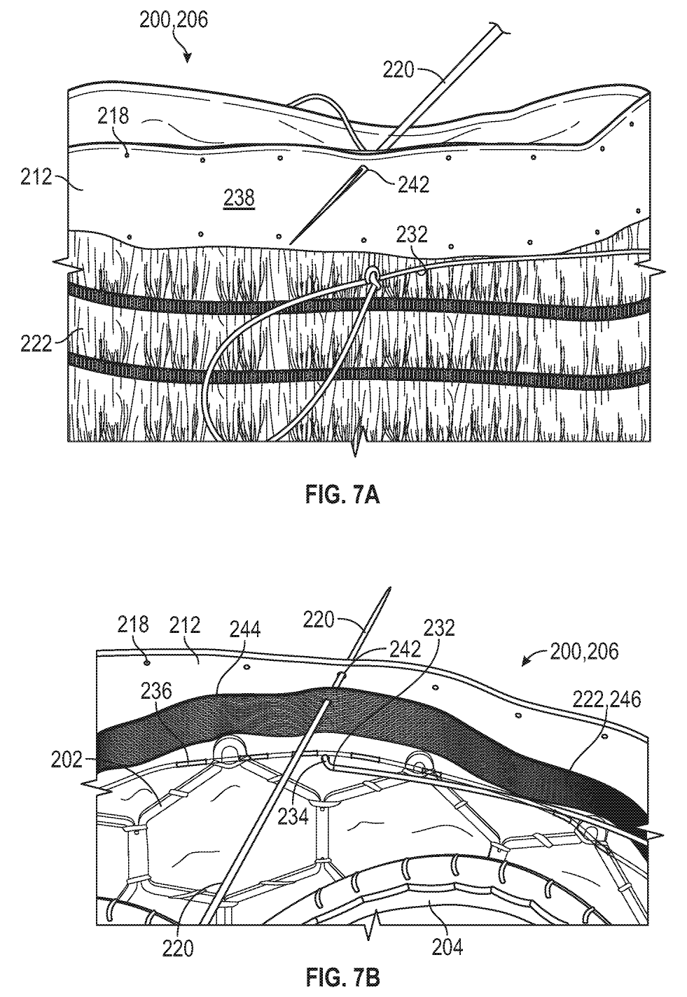

Referring to FIGS. 7A-7B, the needle 220 and suture 232 can then be sewn from the interior of the prosthetic valve 200 to the exterior through the outer covering 222 and the edge covering 212 at a second location 242. As can be seen in FIG. 7B, the second location 242 is located upstream of the first location 234 and is spaced slightly upstream from a free edge 244 of the outer covering 222. In some examples, the position of the second location 242 on the woven portion 246 can be spaced from the free edge 244 of the outer covering 222 by two rows of circumferentially extending yarns of the woven portion.

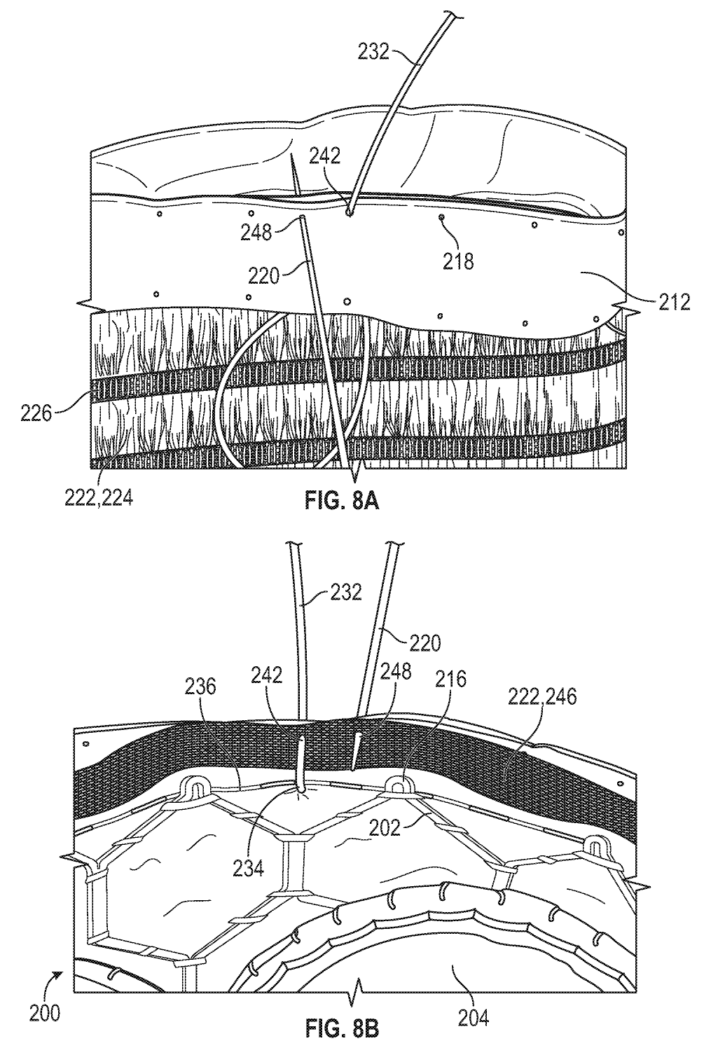

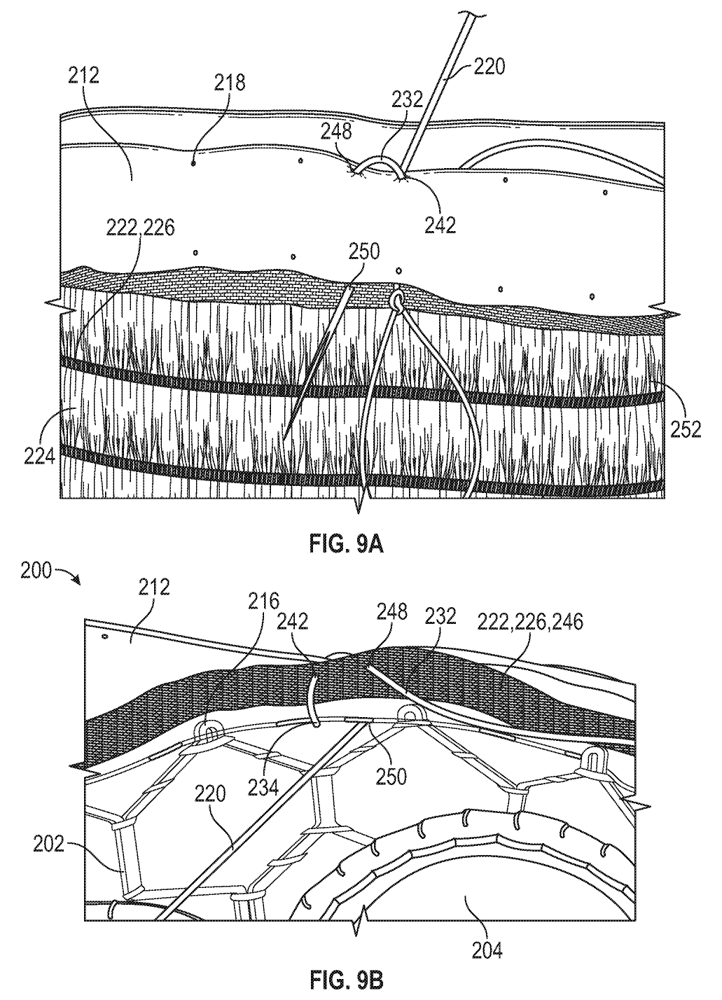

As shown in FIGS. 8A-8B the needle 220 and suture 232 can then be sewn through the edge covering 212 and the outer covering 222 in a direction from the exterior to the interior at a third location 248 laterally adjacent the second location 242. Referring to FIGS. 9A-9B, the needle 220 and suture 232 can then be sewn from the interior to the exterior through the outer covering 222 at a fourth location 250 downstream of the third location 248 and downstream of the suture stitch line 236. The fourth location 250 can be disposed adjacent and upstream of the first float portion 252 at the inflow end of the outer covering 222. The suture does not extend through the edge covering 212 at the fourth location 250. The pattern created by sewing the suture through the first, second, third, and fourth locations can be referred to as a “first stitch” 262. In this way, each subsequent group of four locations can define a subsequent “stitch” 262. For example, the pattern formed by sewing the suture through the fifth, sixth, seventh, and eighth locations can be referred to as the “second stitch.” This pattern can continue circumferentially around the frame 202, as described below.

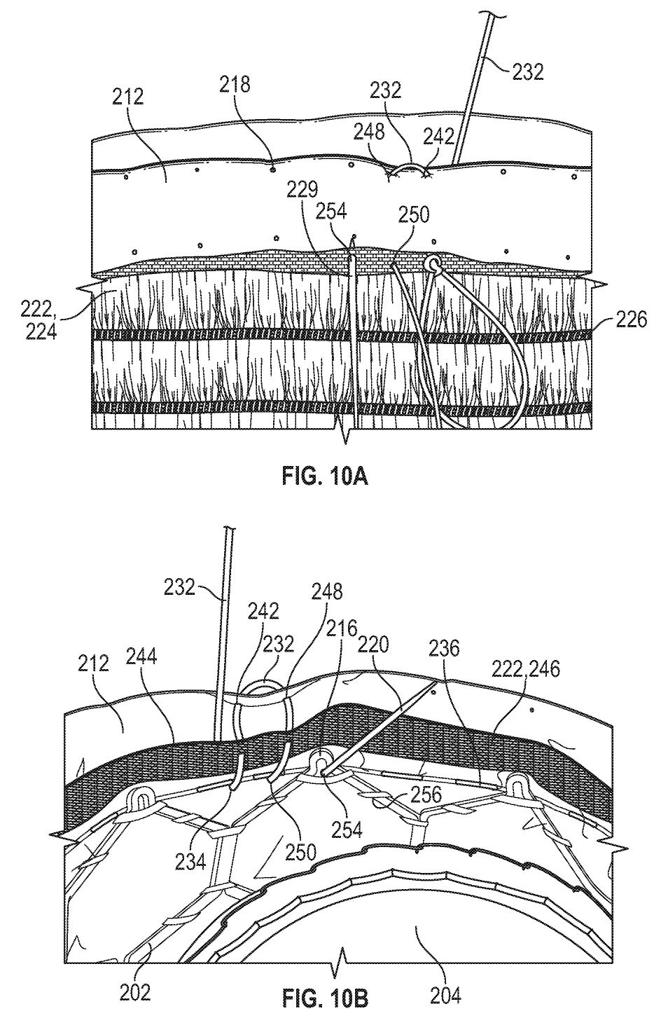

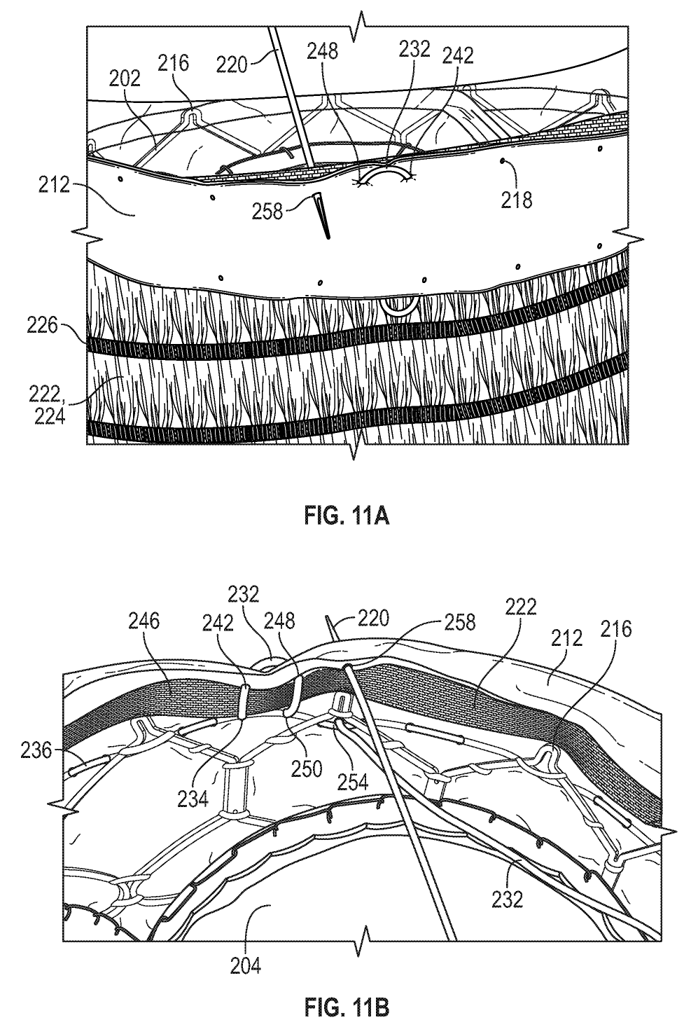

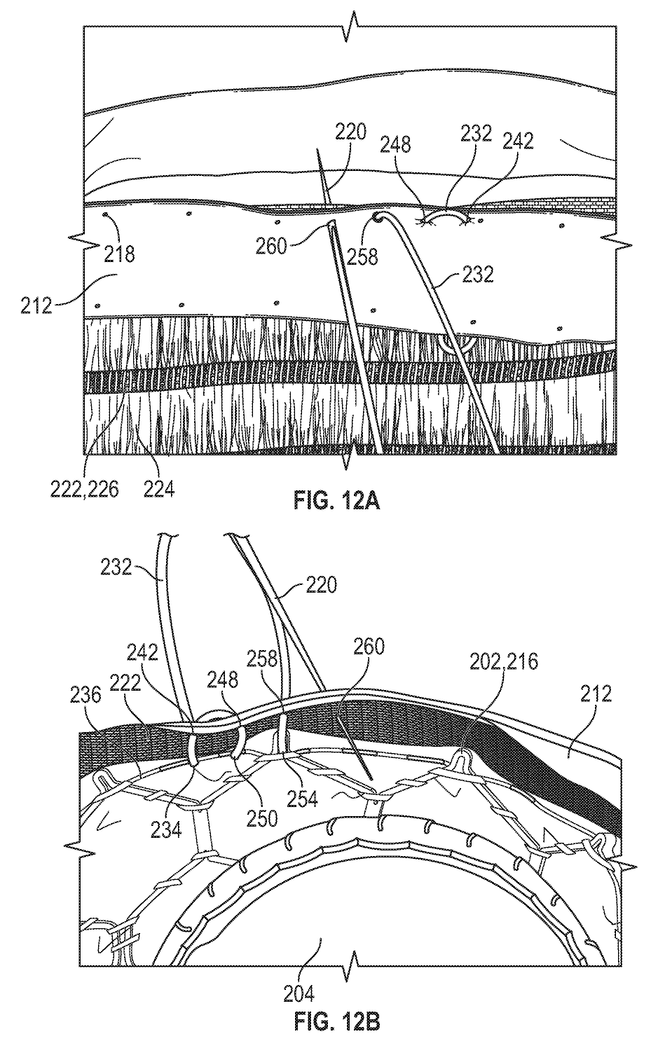

As shown in FIGS. 10A-10B, the needle 220 and suture 232 can then be sewn through the outer covering 222 in a direction from the exterior to the interior at a fifth location 254 laterally adjacent the fourth location 250. In some examples, such as the illustrated example, the fifth location 254 can be disposed upstream of an inflow apex 216 of the frame 202 such that the needle 220 and suture 232 pass through a cell 256 of the frame 202. Referring to FIGS. 11A-11B the needle 220 and suture 232 can then be sewn from the interior to the exterior through the outer covering 222 and the edge covering 212 at a sixth location 258 upstream of the fifth location 254. Referring to FIGS. 12A-12B, the needle 220 and suture 232 can then be sewn from the exterior to the interior through the edge covering 212 and outer covering 222 at a seventh location 260 laterally adjacent the sixth location 258.

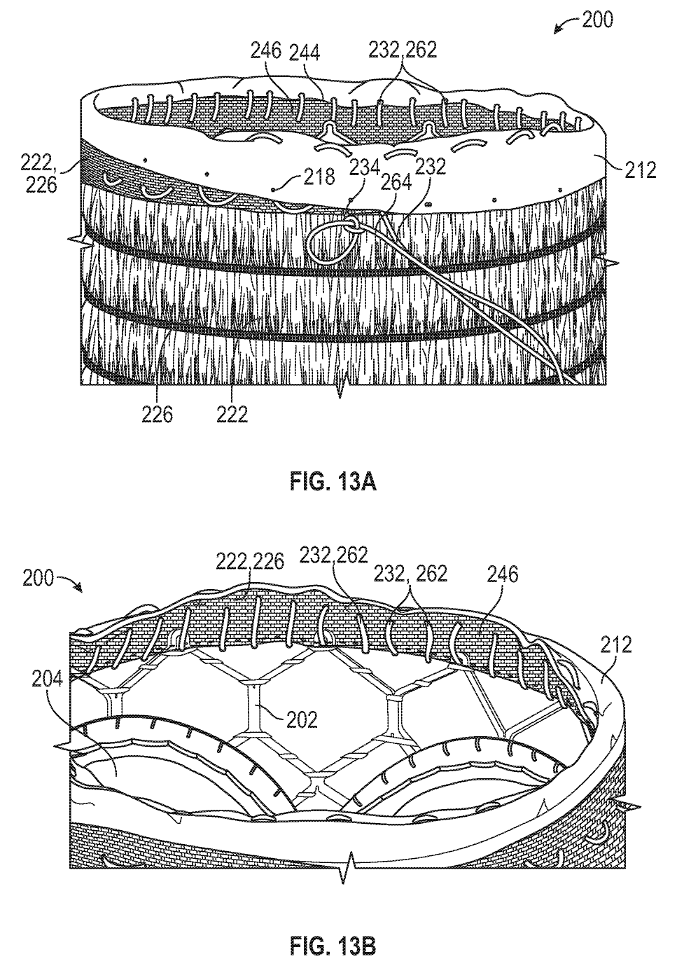

As shown in FIGS. 13A-13B, the previous steps can be repeated until stitches 262 encircle the entire circumference of the prosthetic valve 200 and return to the first location 234 with the needle 220 and suture 232 on the exterior of the frame 202. This location is adjacent the suture tail 264 extending from the first location 234.

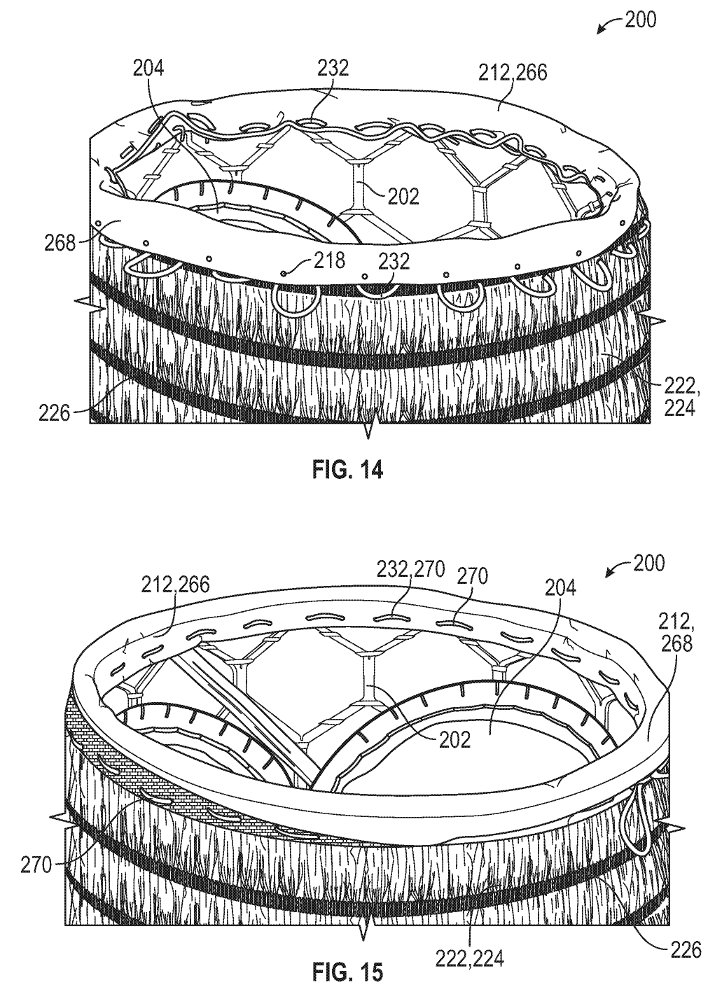

The suture 232 can then be tightened, for example, by applying a pulling force to the portion of the suture 232 coupled to the needle 220 and/or the suture tail 264. As shown in FIG. 14, this causes the edge covering 212 to fold in half lengthwise to define a first layer 266 disposed on the inner surface of the prosthetic valve 200 and a second layer 268 disposed on the outer surface. This method advantageously allows the edge covering 212 to be folded during the suturing/attachment process, rather than when the edge covering 212 is initially disposed on the frame 202. Tightening the suture 232 couples the first layer 266 to the frame 202. In some examples, tightening the suture 232 can also fold the woven portion 246 of the outer covering 222 over the end portion of the frame 202. The assembler can then smooth the first layer 266 and the suture 232, as shown in FIG. 15. The set of stitches extending through the first layer 266 can be referred to collectively as the first set of stitches 270.

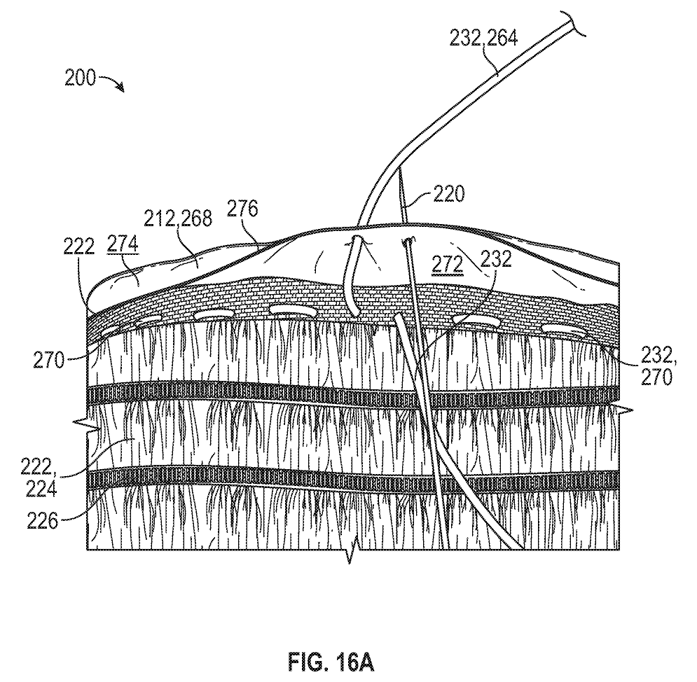

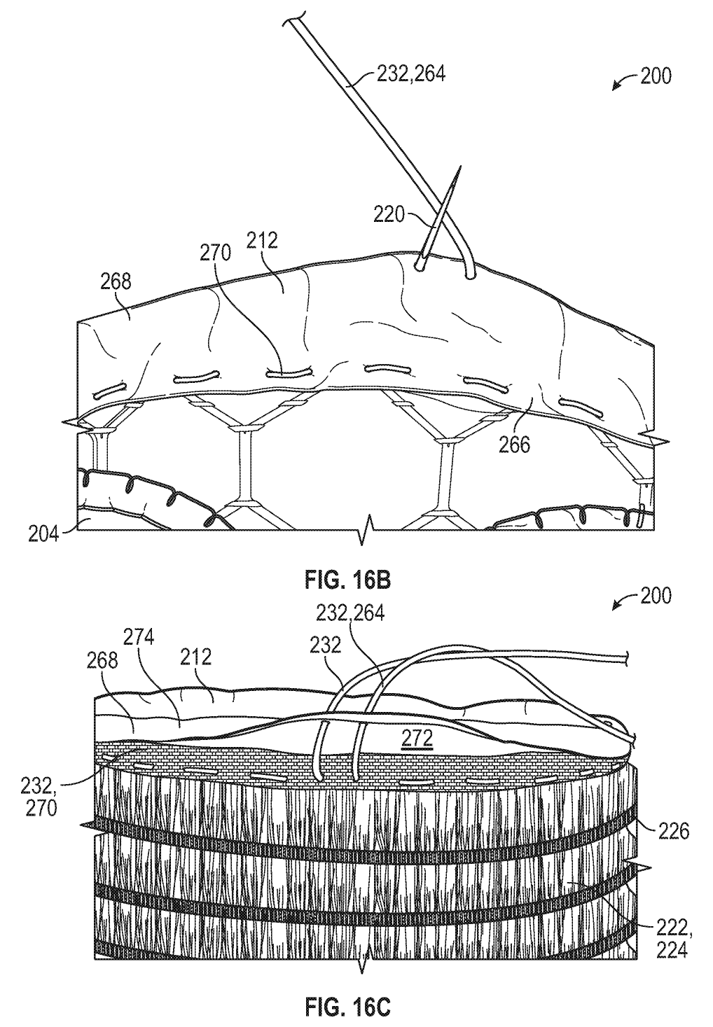

Referring to FIGS. 16A-16C, the needle 220 and suture 232 can then be sewn through the second layer 268 of the edge covering 212 (that is, the layer disposed on the outer surface of the prosthetic valve 200) and the outer covering 222. In some examples, the needle 220 and suture 232 can pass through a preformed aperture 218. In other examples, the needle 220 can create the aperture. The needle 220 and suture 232 can be sewn in a direction from an interior surface 272 of the second layer 268 of the edge covering 212 (e.g., directly adjacent an exterior surface of the outer cover 222) to an exterior surface 274. The suture tail 264 can also be sewn through the edge covering 212 in a direction from the interior surface 272 to the exterior surface 274. In some examples, the suture 232 and suture tail 264 can be sewn through the edge covering 212 at a location approximately 0.5 mm from the outflow edge 276 of the second layer 268 (also referred to as the “second edge” 276, with the outflow edge of the first layer 266 referred to as the “first edge” 278). FIG. 16B shows a view from the interior diameter of the prosthetic valve 200, with the edge covering 212 being partially unfolded such that the second layer 268 is shown above the first layer 266.

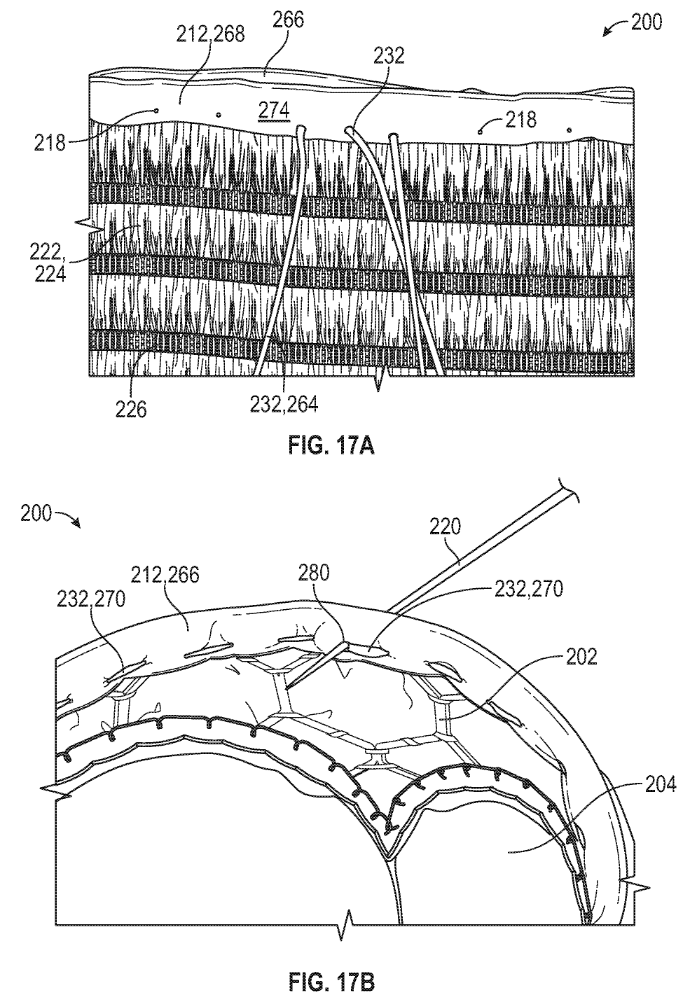

Referring to FIGS. 17A-17B, the second layer 268 can then be folded down such that the interior surface 272 (FIG. 16A) contacts the exterior surface of the outer covering 222. The needle 220 and suture 232 can then be sewn through the second layer 268 of the edge covering 212 (as shown in FIG. 17A), through the outer covering 222, and through the first layer 266 of the edge covering 212, exiting through a respective hole 280 of the first set of stitches 270 as shown in FIG. 17B.

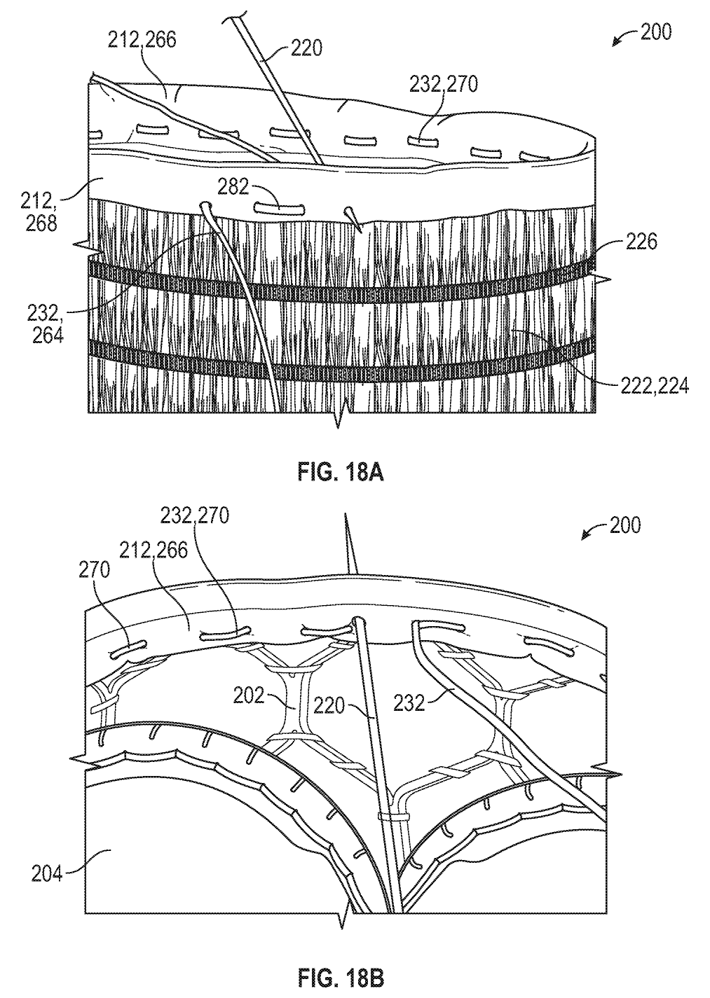

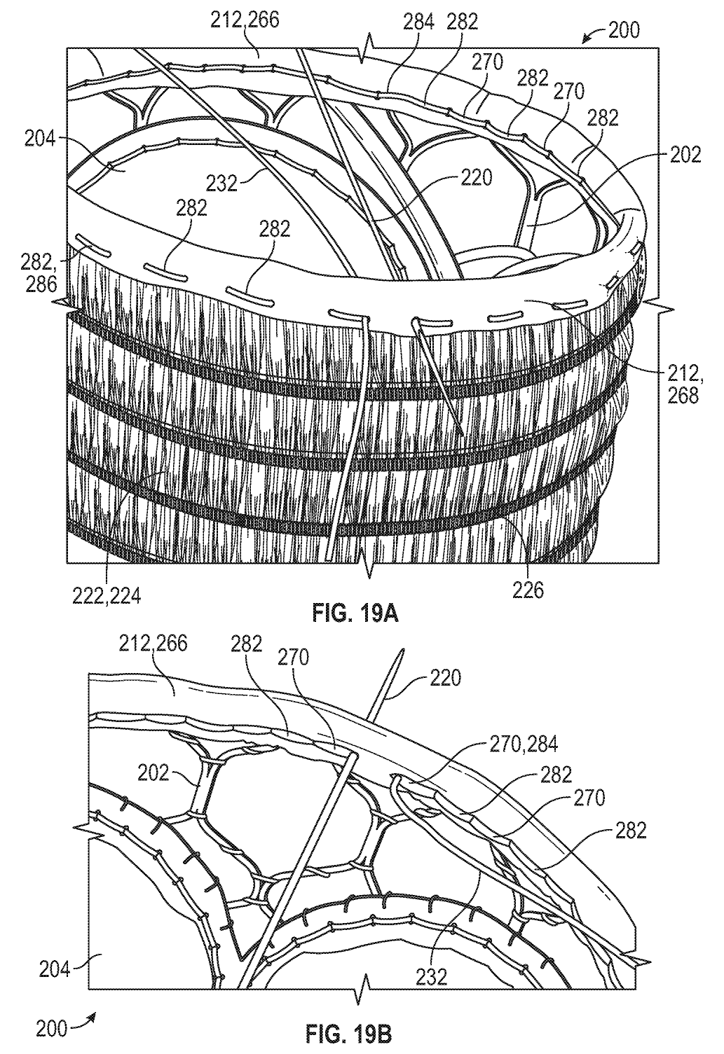

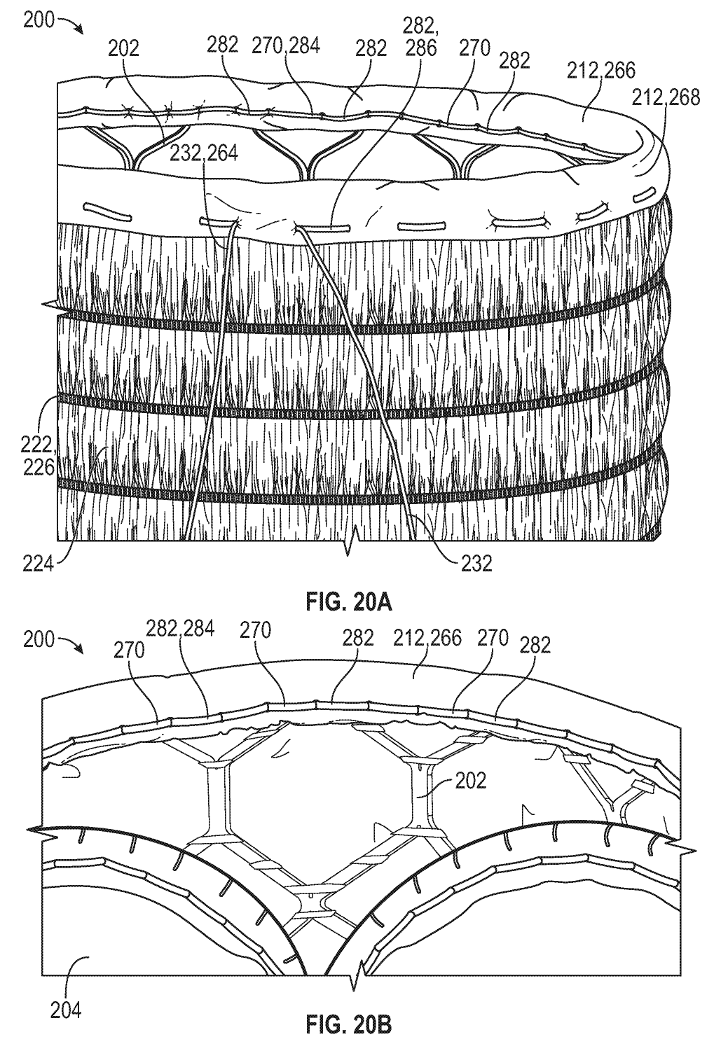

As shown in FIGS. 18A-18B, a running stitch can be used to backfill the first set of stitches with a second set of stitches 282. On the interior diameter of the prosthetic valve 200 the second set of stitches 282 can be configured as backfill stitches, each of which can be sewn between a respective pair of stitches of the first set of stitches 270, as shown in FIG. 18B. On the exterior diameter of the prosthetic valve 200, the second set of stitches 282 can be configured as running stitches with spaces between each stitch such that the second set of stitches 282 appears as a dashed suture line as shown in FIG. 19A. The first suture line 284 on the interior diameter of the prosthetic valve 200 (which comprises the first set of stitches 270 and the second set of stitches 282) appears as a solid line as shown in FIGS. 19A-19B, the second suture line 286 on the exterior diameter of the prosthetic valve (which comprises the second set of stitches 282 only) appears as a dashed line, 286. The running stitch can be repeated until it encircles the entire circumference of the prosthetic valve 200, as shown in FIGS. 20A-20B.

In the illustrated example, the first suture line 284 (solid line) comprising the first set of stitches 270 and the backfill stitches of the second set of stitches 282 is disposed on the interior diameter of the prosthetic valve 200 and the second suture line 286 (dashed line) is disposed on the outer diameter. However, in other examples, the second suture line 286 (dashed line) can be disposed on the inner diameter and the first suture line 284 (solid line) can be disposed on the outer diameter. In some examples, both the inner and outer diameters can comprise solid suture lines or dashed suture lines.

In examples wherein both the inner and outer diameters comprise solid suture lines, in the initial step shown in FIG. 5A/5B, the needle 220 can be sewn through both the outer cover 222 and the edge covering 212 (rather than just the outer cover 222 as shown in FIGS. 5A/5B). This change can be repeated throughout the method of assembly such that the outer stitch line is a solid line rather than a dashed line. In such examples, each stitch coupling the edge covering 212 and the outer cover 222 to the frame extends through both the edge covering 212 and the outer cover 222.

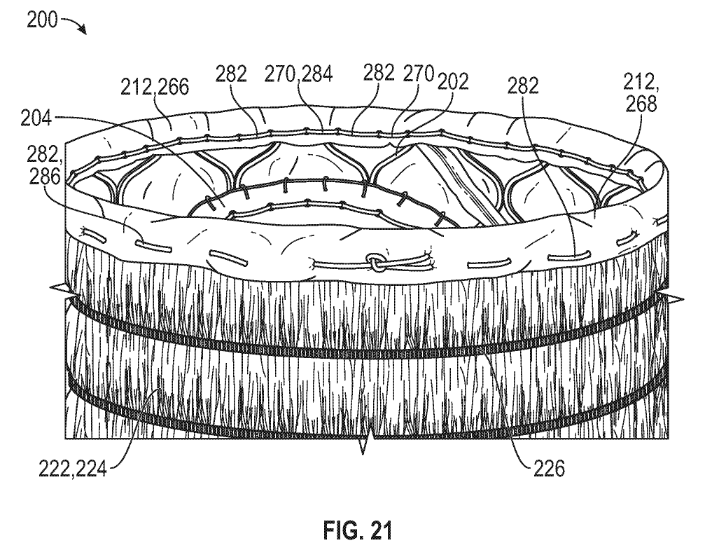

Referring to FIGS. 21, the needle 220 can then be removed, and the suture 232 can be coupled to the suture tail 264 (e.g., using a knot 288 or other coupling method).

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implantable prosthetic device, comprising: a radially expandable and compressible annular frame comprising a first end portion and a second end portion; an outer covering disposed on an outer surface of the frame; and an edge covering disposed on the first end portion of the frame, the edge covering comprising a first layer disposed on an inner facing surface of the frame, and a second layer disposed on the outer surface of the frame; wherein the edge covering is coupled to the frame via a first set of stitches extending through the first layer of the edge covering and the outer covering, and a second set of stitches extending through the first layer of the edge covering, the outer covering, and the second layer of the edge covering.

Example 2. The prosthetic device of any example herein, particularly example 1, wherein the first end portion of the frame is an inflow end portion.

Example 3. The prosthetic device of any example herein, particularly example 1, wherein the first end portion of the frame is an outflow end portion

Example 4. The prosthetic device of any example herein, particularly any one of examples 1-3, wherein the outer covering comprises a plurality of textured portions disposed between a plurality of woven portions.

Example 5. The prosthetic device of any example herein, particularly any one of examples 1-4, wherein the edge covering is a first edge covering, and the prosthetic device further comprises a second edge covering disposed on the second end portion of the frame.

Example 6. The prosthetic device of any example herein, particularly any one of examples 1-5, wherein the edge covering comprises expanded polytetrafluoroethylene (ePTFE).

Example 7. The prosthetic device of any example herein, particularly any one of examples 1-6, wherein the first set of stitches and a portion of the second set of stitches form a first suture line on a radially inwardly facing surface of the edge covering.

Example 8. The prosthetic device of any example herein, particularly example 7, wherein first suture line is a solid suture line.

Example 9. The prosthetic device of any example herein, particularly any one of examples 1-8, wherein a portion of the second set of stitches forms a second suture line on a radially outwardly facing surface of the edge covering.

Example 10. The prosthetic device of any example herein, particularly example 9, wherein the second suture line is a dashed suture line.

Example 11. The prosthetic device of any example herein, particularly any one of examples 1-10, further comprising a valvular structure arranged within the frame, the valvular structure comprising a plurality of leaflets.

Example 12. The prosthetic device of any example herein, particularly any one of examples 1-11, wherein the edge covering comprises a plurality of preformed apertures.

Example 13. A method for securing an edge covering to a frame of a prosthetic valve, comprising: coupling an edge covering to a frame using a first set of stitches extending through an outer covering and a first layer of the edge covering, the outer covering being disposed on an outer surface of the frame, and a second set of stitches extending through a second layer of the edge covering, the outer covering, and the first layer of the edge covering.

Example 14. The method of any example herein, particularly example 13, further comprising after sewing the first set of stitches, tightening the first set of stitches to fold the edge covering in half lengthwise forming the first and second layers of the edge covering.

Example 15. The method of any example herein, particularly any one of examples 13-14, wherein the first layer of the edge covering is disposed on an inner surface of the frame and the second layer of the edge covering is disposed on an outer surface of the frame.

Example 16. The method of any example herein, particularly example 15, wherein the outer covering is disposed between the second layer of the edge covering and the outer surface of the frame.

Example 17. A method of assembling a prosthetic valve, comprising: disposing an outer covering on a radially outer facing surface of a radially expandable and compressible frame; sewing a suture through the outer covering at a first location; disposing an edge covering around a first end portion of the frame partially over the outer covering; sewing the suture through the outer covering and the edge covering at a second location upstream of the first location; sewing the suture through the edge covering and the outer covering at a third location laterally adjacent the second location; sewing the suture through the outer covering at a fourth location downstream of the third location to form a first stitch; sewing the suture into a second stitch laterally adjacent to the first stitch; sewing the suture into additional stitches around a circumference of the frame to form a first set of stitches that includes the first stitch, the second stitch, and the additional stitches; and tightening the suture to secure a first layer of the edge covering to a radially inner facing surface of the frame.

Example 18. The method of any example herein, particularly example 17, further comprising: sewing the suture through a second layer of the edge covering; folding the second layer such that it contacts the outer covering; sewing the suture through the second layer of the edge covering, the outer covering, and the first layer such that it extends through a respective hole of the first set of stitches; and backfilling the first set of stitches with a second set of stitches by sewing the suture through both layers of the edge covering and the outer covering to form a first suture line on a radially inwardly facing surface of the edge covering and a second suture line on a radially outwardly facing surface of the edge covering.

Example 19.The method of any example herein, particularly example 18, wherein the first suture line is a solid suture line and the second suture line is a dashed suture line.

Example 20.The method of any example herein, particularly any one of examples 18-19, wherein the first suture line comprises the first set of stitches and a portion of the second set of stitches.

Example 21.The method of any example herein, particularly any one of examples 18-19, wherein the second suture line comprises a portion of the second set of stitches.

Example 22.The method of any example herein, particularly any one of examples 17-21, further comprising coupling a first end of the suture to a second end of the suture.

Example 23.The method of any example herein, particularly any one of examples 17-22, wherein the edge covering is a first edge covering and is disposed on an inflow end of the frame, and wherein the method further comprises coupling a second edge covering to an outflow end of the frame.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. An implantable prosthetic device, comprising:
a radially expandable and compressible annular frame comprising a first end portion and a second end portion;
an outer covering disposed on an outer surface of the frame; and
an edge covering disposed on the first end portion of the frame, the edge covering comprising a first layer disposed on an inner facing surface of the frame, and a second layer disposed on the outer surface of the frame;
wherein the edge covering is coupled to the frame via a first set of stitches extending through the first layer of the edge covering and the outer covering, and a second set of stitches extending through the first layer of the edge covering, the outer covering, and the second layer of the edge covering.

2. The prosthetic device of claim 1, wherein the first end portion of the frame is an inflow end portion.

3. The prosthetic device of claim 1, wherein the first end portion of the frame is an outflow end portion.

4. The prosthetic device of claim 1, wherein the outer covering comprises a plurality of textured portions disposed between a plurality of woven portions.

5. The prosthetic device of claim 1, wherein the edge covering is a first edge covering, and the prosthetic device further comprises a second edge covering disposed on the second end portion of the frame.

6. The prosthetic device of claim 1, wherein the first set of stitches and a portion of the second set of stitches form a first suture line on a radially inwardly facing surface of the edge covering.

7. The prosthetic device of claim 6, wherein first suture line is a solid suture line.

8. The prosthetic device of claim 1, wherein a portion of the second set of stitches forms a second suture line on a radially outwardly facing surface of the edge covering.

9. The prosthetic device of claim 8, wherein the second suture line is a dashed suture line.

10. The prosthetic device of claim 1, further comprising a valvular structure arranged within the frame, the valvular structure comprising a plurality of leaflets.

11. The prosthetic device of claim 1, wherein the edge covering comprises a plurality of preformed apertures.

12. A method for securing an edge covering to a frame of a prosthetic valve, comprising:
coupling an edge covering to a frame using a first set of stitches extending through an outer covering and a first layer of the edge covering, the outer covering being disposed on an outer surface of the frame, and a second set of stitches extending through a second layer of the edge covering, the outer covering, and the first layer of the edge covering.

13. The method of claim 12, further comprising after sewing the first set of stitches, tightening the first set of stitches to fold the edge covering in half lengthwise forming the first and second layers of the edge covering.

14. The method of claim 12, wherein the first layer of the edge covering is disposed on an inner surface of the frame and the second layer of the edge covering is disposed on an outer surface of the frame.

15. The method of claim 14, wherein the outer covering is disposed between the second layer of the edge covering and the outer surface of the frame.

16. A method of assembling a prosthetic valve, comprising:
disposing an outer covering on a radially outer facing surface of a radially expandable and compressible frame;
sewing a suture through the outer covering at a first location;
disposing an edge covering around a first end portion of the frame partially over the outer covering;
sewing the suture through the outer covering and the edge covering at a second location upstream of the first location;
sewing the suture through the edge covering and the outer covering at a third location laterally adjacent the second location;
sewing the suture through the outer covering at a fourth location downstream of the third location to form a first stitch;
sewing the suture into a second stitch laterally adjacent to the first stitch;
sewing the suture into additional stitches around a circumference of the frame to form a first set of stitches that includes the first stitch, the second stitch, and the additional stitches; and
tightening the suture to secure a first layer of the edge covering to a radially inner facing surface of the frame.

17. The method of claim 16, further comprising:
sewing the suture through a second layer of the edge covering;
folding the second layer such that it contacts the outer covering;
sewing the suture through the second layer of the edge covering, the outer covering, and the first layer such that it extends through a respective hole of the first set of stitches; and
backfilling the first set of stitches with a second set of stitches by sewing the suture through both layers of the edge covering and the outer covering to form a first suture line on a radially inwardly facing surface of the edge covering and a second suture line on a radially outwardly facing surface of the edge covering.

18. The method of claim 17, wherein the first suture line is a solid suture line and the second suture line is a dashed suture line.

19. The method of claim 17, wherein the first suture line comprises the first set of stitches and a portion of the second set of stitches.

20. The method of claim 17, wherein the second suture line comprises a portion of the second set of stitches.
